(19) United States
(12) Patent Application Publication
(10) Pub. No.: US 2021/0076971 A1
(43) Pub. Date: Mar. 18, 2021

(54) Monitoring Vital Sign of Occupant in a Vehicle Seat

(71) Applicant: Infineon Technologies AG, Neubiberg (DE)

(72) Inventors: Daniel Oloumi, Villach (AT); Matteo Bassi, Villach (AT)

(21) Appl. No.: 16/570,764

(22) Filed: Sep. 13, 2019

(51) Int. Cl. A61B 5/05; A61B 5/0205; A61B 5/00; A61B 5/11; G01S 13/88; B60N 2/24

(52) U.S. Cl. CPC A61B 5/05; A61B 5/0205; A61B 5/0002; A61B 5/742; A61B 5/021; A61B 5/1116; A61B 5/486; G01S 13/88; B60N 2/24; A61B 5/7475

(57) ABSTRACT

A method for measuring vital signs of an occupant in a vehicle seat is provided. In an embodiment, a sensor includes a transmission circuitry, a reception circuitry, and a controller. The transmission circuitry is configured to transmit a radar beam to an occupant in a vehicle seat. The reception circuitry is configured to receive a reflected signal. The reflected signal being a reflection of the radar beam from the occupant. The controller is configured to determine data associated with a vital sign of the occupant based on the reflected signal.

### TECHNICAL FIELD

The present disclosure relates generally to electronic devices, and, in particular embodiments, to systems and methods for monitoring a vital sign of an occupant in a vehicle seat.

### BACKGROUND

One cause for vehicle accidents is an incapacitation of the driver of a vehicle due to heart and respiratory problems. With advancements in self-driving (i.e., autonomous) vehicles and the growth in vehicle-to-vehicle (V2V), vehicle to infrastructure (V2I), and vehicle to everything (V2X) technologies, opportunities exist to prevent and seek assistance in the event of such an occurrence.

Typically, the monitoring of changes in the size and movement of the rib cage can provide fairly accurate heartbeat and respiratory measurements of the driver. Existing solutions to monitor vital signs of the driver include sensors mounted in the steering wheel, dashboard, rear-view mirror, vehicle ceiling, or on the seat belt of the vehicle. In each case, the sensor is prone to blockage, inconsistencies in measurements, and frequent errors due to the movement of the occupant and the relative positioning of the driver to the sensor.

A need therefore exists for improved systems and methods for monitoring a vital sign of an occupant in a vehicle seat.

### SUMMARY

Technical advantages are generally achieved by embodiments of this disclosure, which describe systems and methods for monitoring a vital sign of an occupant in a vehicle seat.

A first aspect relates to a sensor that includes a transmission circuitry, a reception circuitry, and a controller. The transmission circuitry is configured to transmit a radar beam to an occupant in a vehicle seat. The reception circuitry is configured to receive a reflected signal—a reflection of the radar beam from the occupant. The controller is configured to determine data associated with a vital sign of the occupant based on the reflected signal.

In a first implementation form of the sensor according to the first aspect, the sensor further includes an interface circuitry configured to transmit data from and receive data by the sensor.

In a second implementation form of the sensor, according to the first aspect as such or any preceding implementation form of the first aspect, the controller is further configured to transmit the data associated with the vital sign of the occupant to an external component using the interface circuitry.

In a third implementation form of the sensor, according to the first aspect as such or any preceding implementation form of the first aspect, the vital sign is a heart rate, a respiratory rate, or a blood pressure rate of the occupant, or a combination thereof.

In a fourth implementation form of the sensor, according to the first aspect as such or any preceding implementation form of the first aspect, the sensor further includes a non-transitory memory storage configured to store the data associated with the vital sign of the occupant.

In a fifth implementation form of the sensor, according to the first aspect as such or any preceding implementation form of the first aspect, the reception circuitry includes a plurality of antennas arranged in an array configuration within a seat portion of a vehicle seat assembly.

In a sixth implementation form of the sensor, according to the first aspect as such or any preceding implementation form of the first aspect, the transmission circuitry includes a plurality of antennas arranged in an array configured within a seat portion of a vehicle seat assembly.

In a seventh implementation form of the sensor, according to the first aspect as such or any preceding implementation form of the first aspect, the controller is further configured to calibrate the transmission circuitry to direct the radar beam to a heart location on a rib cage of the occupant.

In an eight implementation form of the sensor, according to the first aspect as such or any preceding implementation form of the first aspect, the transmission circuitry is configured to direct the radar beam to a posterior upper limb portion of the occupant.

In a ninth implementation form of the sensor, according to the first aspect as such or any preceding implementation form of the first aspect, the controller is further configured to determine a position of the occupant in the vehicle seat based on an estimated time delay, an amplitude, or a phase of the reflected signal and, based thereon, to adjust one or more phase shifters in the transmission circuitry.

In a tenth implementation form of the sensor, according to the first aspect as such or any preceding implementation form of the first aspect, the controller is further configured to determine a position of the occupant in the vehicle seat based on an estimated time delay, an amplitude, or a phase of the reflected signal and, based thereon, to adjust one or more phase shifters in the reception circuitry.

In an eleventh implementation form of the sensor, according to the first aspect as such or any preceding implementation form of the first aspect, the radar beam includes a continuous RF signal, a pulsed RF signal, a modulated RF signal, or a combination thereof.

A second aspect relates to a method that includes transmitting, by a transmission circuitry, a radar beam to an occupant in a vehicle seat; receiving, by a reception circuitry, a reflected signal that is a reflection of the radar beam from the occupant; and determining, by a controller, data associated with a vital sign of the occupant based on the reflected signal.

In a first implementation form of the method according to the second aspect, the method further includes transmitting, using an interface circuitry, the data associated with the vital sign of the occupant to an external component.

In a second implementation form of the method, according to the second aspect as such or any preceding implementation form of the second aspect, the method further includes analyzing, storing, or displaying the data associated with the vital sign of the occupant, or a combination thereof.

In a third implementation form of the method, according to the second aspect as such or any preceding implementation form of the second aspect, the method further includes calibrating the transmission circuitry to direct a direction of the radar beam to a rib cage of the occupant.

In a fourth implementation form of the method, according to the second aspect as such or any preceding implementation form of the second aspect, the method further includes determining a position of the occupant in the vehicle seat based on an estimated time delay, an amplitude, or a phase of the reflected signal and, based thereon, adjusting one or more phase shifters in the transmission circuitry.

In a fifth implementation form of the method, according to the second aspect as such or any preceding implementation form of the second aspect, the method further includes determining a position of the occupant in the vehicle seat based on an estimated time delay, an amplitude, or a phase of the reflected signal and, based thereon, adjusting one or more phase shifters in the reception circuitry.

In a sixth implementation form of the method, according to the second aspect as such or any preceding implementation form of the second aspect, the method further includes determining a position of the occupant in the vehicle seat based on an estimated time delay, an amplitude, or a phase of the reflected signal and, based thereon, providing a feedback to the occupant to adjust his/her posture for improved measurement accuracy.

In a seventh implementation form of the method, according to the second aspect as such or any preceding implementation form of the second aspect, the vital sign is a first vital sign, the method further includes receiving, from an external source, a second vital sign of the occupant and, based thereon, determining a combined data associated with the second vital sign and the first vital sign.

In an eight implementation form of the method, according to the second aspect as such or any preceding implementation form of the second aspect, the radar beam includes a continuous RF signal, a pulsed RF signal, a modulated RF signal, or a combination thereof.

A third aspect relates to a vehicle seat that includes a seat portion, a non-transitory memory storage, a plurality of antennas, a transmission circuitry, a reception circuitry, and a controller. The plurality of antennas are configured in an array configuration within the seat portion. The transmission circuitry is coupled with the plurality of antennas. The transmission circuitry is configured to transmit, over one or more of the plurality of antennas, a radar beam to an occupant in the vehicle seat. The reception circuitry is coupled with the plurality of antennas. The reception circuitry is configured to receive, over one or more of the plurality of antennas, a reflected signal that is a reflection of the radar beam from the occupant. The controller is configured to: (1) determine data associated with a vital sign of the occupant based on the reflected signal; and (2) store the data associated with the vital sign of the occupant in the non-transitory memory storage.

In a first implementation form of the vehicle seat according to the third aspect, the controller is further configured to determine occupancy of the vehicle seat by the occupant and, based thereon, transmitting the radar beam.

In a second implementation form of the vehicle seat, according to the third aspect as such or any preceding implementation form of the third aspect, a plurality of N number of antennas are arranged in a generic pattern configuration, wherein an M number of antennas are transmitting antennas and an (N−M) number of antennas are receiving antennas, N being a positive integer greater than 1 and M being a positive integer.

In a third implementation form of the vehicle seat, according to the third aspect as such or any preceding implementation form of the third aspect, the plurality of antennas are arranged in a generic pattern configuration, wherein each antenna is a transmitting antenna and a receiving antenna.

In a fourth implementation form of the vehicle seat, according to the third aspect as such or any preceding implementation form of the third aspect, the vehicle seat further includes a plurality of circulators, wherein the transmission circuitry and the reception circuitry are coupled to each antenna using a respective circulator.

In a fifth implementation form of the vehicle seat, according to the third aspect as such or any preceding implementation form of the third aspect, the controller is further configured to determine a change in a posture of the occupant in the vehicle seat and, based thereon, providing feedback, using an external display on a vehicle that the vehicle seat is mounted, for the occupant to adjust the posture of the occupant for improved vital sign measurements.

In a sixth implementation form of the vehicle seat, according to the third aspect as such or any preceding implementation form of the third aspect, the controller is further configured to provide, to an external component on a vehicle that the vehicle seat is mounted, data associated with the vital sign to be displayed by the external component.

In a seventh implementation form of the vehicle seat, according to the third aspect as such or any preceding implementation form of the third aspect, the radar includes a continuous RF signal, a pulsed RF signal, a modulated RF signal, or a combination thereof.

Embodiments can be implemented in hardware, software, or in any combination thereof. A computer program can perform the operations hereinabove. A device can be programmably-arranged to perform the computer program.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1A is an illustration of an occupant positioned within a vehicle seat;

FIG. 1B is an illustration of an occupant positioned within a vehicle seat outfitted with a sensor to measure vital signs of the occupant;

FIG. 2A is a combined schematic and block diagram of an embodiment sensor used to measure a vital sign of the occupant;

FIG. 2B is a combined schematic and block diagram of another embodiment sensor used to measure a vital sign of the occupant;

FIGS. 3A-D illustrate several non-limiting embodiment arrangements of antenna elements within a vehicle seat;

FIG. 4 is a flowchart of an embodiment method for calibrating a sensor, as may be performed by a controller of the sensor; and

FIG. 5 is a flowchart of an embodiment method for collecting data associated with a vital sign measurement of an occupant.

### DETAILED DESCRIPTION OF ILLUSTRATIVE EMBODIMENTS

This disclosure provides many applicable inventive concepts that can be embodied in a wide variety of specific contexts. The specific embodiments are merely illustrative of specific configurations and do not limit the scope of the claimed embodiments. Features from different embodiments may be combined to form further embodiments unless noted otherwise. Variations or modifications described with respect to one of the embodiments may also be applicable to other embodiments. Further, it should be understood that various changes, substitutions, and alterations can be made herein without departing from the spirit and scope of this disclosure as defined by the appended claims.

The description below illustrates the various specific details to provide an in-depth understanding of several example embodiments according to the description. The embodiments may be obtained without one or more of the specific details, or with other methods, components, materials and the like. In other cases, known structures, materials, or operations are not shown or described in detail so as not to obscure the different aspects of the embodiments. References to “an embodiment” in this description indicate that a particular configuration, structure, or feature described in relation to the embodiment is included in at least one embodiment. Consequently, phrases such as “in one embodiment” that may appear at different points of the present description do not necessarily refer exactly to the same embodiment. Furthermore, specific formations, structures, or features may be combined in any appropriate manner in one or more embodiments.

While the inventive aspects are described primarily in the context of monitoring a vital sign of an occupant in a vehicle seat, it should also be appreciated that these inventive aspects are also applicable across any variation of apparatuses that provide for the transmission of signals to a back of an occupant, reception of a reflection of the signal, and determination of vital signs of the occupant.

Embodiments of this disclosure provide techniques for contactless monitoring of a vital sign of an occupant in a vehicle seat. In an embodiment, a sensor includes a transmission circuitry, reception circuitry, and a controller. A radar beam is transmitted to the occupant using the transmission circuitry. The reflection of the radar beam from the occupant is received by the reception circuitry in the form of a reflected signal. The vital signs of the occupant are then measured based on the reflected signal. The vital sign can be one or more of heart rate, a respiratory rate, or a blood pressure rate of the occupant.

The reception circuitry and the transmission circuitry may each include multiple antennas and phase shifters. The multiple antennas may be arranged in an array configuration within a seat portion of the vehicle seat assembly. The antennas may be positioned in various locations of the vehicle seat assembly. As an example, the antennas may be located on the front, side, back, inside, above, or a combination thereof, of a seat back portion of the vehicle seat assembly. The antennas may be flexible and conform to the general outline of the seat portion. The antennas may be arranged in a variety of configurations, such as a star arrangement, matrix arrangement, a circle type arrangement, a diamond configuration, or others.

In various embodiments, N number of antennas are arranged in a generic pattern configuration within the vehicle seat. In this embodiment, M number of antennas are transmitting antennas and an (N−M) number of antennas are receiving antennas, where N is a positive integer greater than 1 and M is a positive integer. In embodiments, each antenna may be a transmitting and a receiving antenna. The sensor may include one or more circulators used to couple the transmission and reception circuitry to a corresponding antenna element. In some embodiments, multiple antennas may be used to implement directional beams using phased array techniques. A multiple-input multiple-output (MIMO) configuration with multiple chipsets can be used to perform coherent and non-coherent signal processing.

Aspects of this disclosure provide embodiment calibration and measurement procedures for monitoring of a vital sign of the occupant. In particular, a calibration procedure is introduced to remove ambient effects and target a reconfigurable radar beam to a location of the heart within the ribcage of the occupant. In an embodiment, the controller determines a position of the occupant in the vehicle seat and a movement of the rib cage based on an estimated time delay, an amplitude, or a phase of the reflected signal. In embodiments, the position of the occupant and the movement of the rib cage is determined based on a time delay measurement of the reflected signal. The controller calibrates the phase shifters in the transmission and reception circuitries to direct a radar beam to a targeted location of the occupant. As such, the radar beam may be a reconfigurable radar beam. In an embodiment, the radar beam is directed to a heart location on the rib cage of the occupant. In another embodiment, the radar beam is directed to the general direction of the rib cage of the occupant. In another embodiment, the radar beam is directed to a posterior upper limb portion of the occupant. In embodiments, the sensor may communicate to the occupant, for example through an interface, to adjust his or her posture for improved measurement accuracy of the vital signs. In another embodiment, the sensor may detect the occupancy of the vehicle seat and then initiate the process of collecting, storing, analyzing, displaying and/or communicating the data associated with the vital sign. By using beam steering, the radar beam may be directed in a particular direction. This may be used, for example, to direct the radar beam toward fine veins and arteries in order to perform more accurate and precise vital signal measurements. In various embodiments, ambient effects that can introduce errors to the measurement of the vital sign are removed during the calibration process. This advantageously provides a more reliable and more consistent measurement of the vital sign.

The sensor may also include an interface circuitry that is used to transmit and receive data. In some embodiments, the controller transmits the data associated with the vital sign to an external component using, for example, the interface circuitry. In some embodiments, the sensor may also include a non-transitory memory storage used to store the data associated with the vital sign of the occupant. In some embodiments, the data associated with the vital sign measurements are displayed on a dashboard of the vehicle. In other embodiments, the data is transmitted to an external component, for example a cellular phone or a computer, which can display or provide analysis of the collected data. The data may be analyzed internally to the sensor or communicated with an external system using, for example, the interface circuitry.

In various embodiments, the data collected by the sensor may be supplemented by data collected from other sensors. A combined data set based on the analyses of the vital signs collected from the sensor and one or more external sources may be used to provide a comprehensive data set to the occupant. In embodiments, the external source may be a cellular phone, and/or a wearable electronic device (e.g., smartwatch, armband, earpiece, chest strap, etc.). These and other details are discussed in greater detail below.

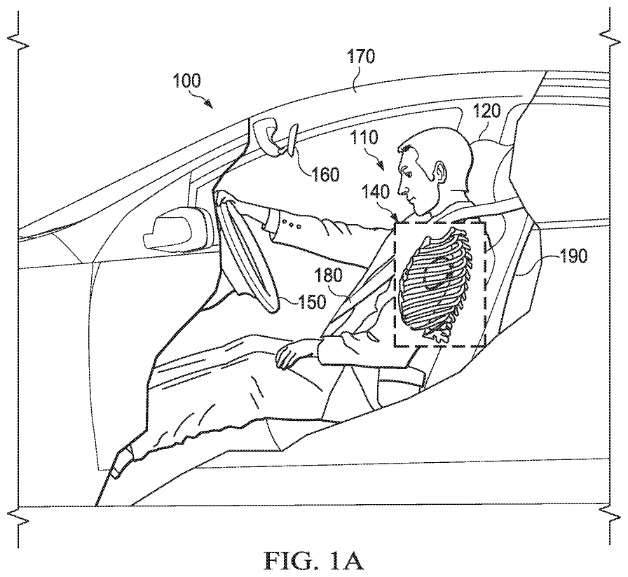

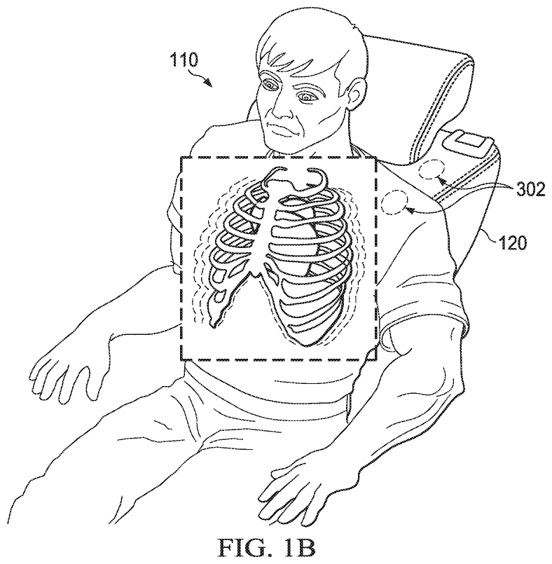

FIG. 1A illustrates an occupant 110 positioned within a vehicle seat 120 of a vehicle 100. It is advantageous to monitor a vital sign of the occupant 110, as constant monitoring of the vital signs may be helpful in minimizing or preventing accidents due to a heart failure, for example while driving. The vital sign may be a heart rate, a respiratory rate, or a blood pressure rate of the occupant 110, or a combination thereof. The back and forth movements of the rib cage 140 can indicate the breathing rate and heart beat frequency of the occupant 110. The movement of the rib cage 140 can be measured using a contactless sensor, such as a radar signal. This is typically done by transmitting a radio frequency (RF) signal using a radar to the rib cage 140 and collecting the reflected signal. The reflected signal is analyzed and data associated with the vital sign is calculated using the time delay, amplitude, or a phase difference in reference to the transmitted signal. In some embodiments, a time delay measurement is used to measure the relative location at a moment of time, which over time may be used to determine the heartbeat frequency or a respiratory rate.

In various embodiments of this disclosure, the sensor is located on the vehicle seat 120, and, in particular embodiments, on the back seat portion 190 of the vehicle seat 120. In embodiments, the sensor is located within or external to the vehicle seat 120. These and other embodiments provide systems and methods for monitoring a vital sign of the occupant 110 that advantageously provide a near clear path between the sensor and the rib cage 140 of the occupant 110.

The embodiments of this disclosure provide a more reliable and accurate set of measurements mainly due to the positioning of the sensors relative to the occupant 110 and potential motion of the body relative to the sensors. These advantages are in contrast to previous implementations that have sensors mounted in the dashboard or the steering wheel 150, rear-view mirror 160, ceiling 170, or the seat belt 180 of the vehicle 100.

As an example, sensors located in the dashboard or the steering wheel 150 may become obstructed by movements of various body parts (e.g., hand and arm) of the occupant 110. As another example, sensors located in the rear-view mirror 160 or ceiling 170 may lack a proper angle to accurately measure movements of the rib cage 140. Additionally, the head of the occupant may obstruct these measurements. In yet another example, sensors located in the seat belt 180 simultaneously move with the rib cage 140 and do not have a different frame of reference. Further, as the sensor is attached to the occupant 110 and forms around the occupant 110, the location of the sensor needs to be modified based on a change in the shape and size of the occupant.

Additionally, aspects of this disclosure provide a system and method that uses a reconfigurable radar beam to adjust the direction of RF signals to the heart of the occupant 110 within the rib cage 140 and to receive reflected signals of the RF transmission from the occupant 110. This is mainly done by continuous monitoring and sensing of the displacement of the upper portion of the body relative to the direction at which the reconfigurable radar beam is pointed.

The inventive concepts in the present application may be additionally advantageous with the advancements in autonomous vehicles. As an example, a fully or semi-automated process may be configured within the vehicle, in which a sensor is in constant or near constant communication with a vehicle computer. The sensor may communicate the data to the vehicle computer. The vehicle computer may then determine that a measurement, corresponding to a vital sign, has exceeded safe conditions and an emergency situation may be triggered. Alternatively, a controller of the sensor may determine that the measurement, corresponding to the vital sign, has exceeded safe conditions. The sensor may communicate the emergency situation to the vehicle computer. In either case, the vehicle computer may reroute or steer the vehicle to the nearest emergency care unit.

In an embodiment, a semi-autonomous vehicle may determine the best time or moment to yield control back to the driver based on, for example, the heartbeat and respiratory measurements. The measurements may be used as an indication for the drivers' level of awareness and stress. Such a solution may be advantageous to provide methods in exchanging control between autonomous and driver mode. In other words, the proposed solutions may be used to gauge the drivers' level of readiness and/or capability to retain vehicle control. It may be likewise advantageous to monitor the health and stress level of the driver.

The progressions in vehicle to vehicle (V2V), vehicle to infrastructure (V2I), and vehicle to everything (V2X) technologies additionally provides opportunities for communication exchange with external databases and/or cloud based analysis of the vital signs. Additionally, the networked vehicle computer, a cellular phone external to the vehicle, or the cloud based computer may communicate the data to the emergency care unit prior to the arrival of the occupant and to provide relevant information needed to overcome the emergency situation.

Referring back to FIG. 1A, the selective placement of the sensor within or on the vehicle seat 120, relative to the occupant 110, allows for a reduction or minimization of obstructions caused by movements of major body parts (e.g., hands and arms). The obstructions caused by the body parts may block or disrupt the collection of the transmission and reception of the a pulse targeted at the rib cage 140.

FIG. 1B illustrates an occupant 110 positioned within a vehicle seat 120 used to measure vital signs of the occupant 110. In an embodiment, the reflected signal may have a range profile of R+R₁+R₂. In this embodiment, R is the roundtrip distance of the RF signal to and back from the body. R1 and R2 contain variations in movement associated with the breathing and the heartbeat of the occupant 110 exposed to the RF signal.

In an embodiment, and as disclosed further herein, a sensor may include, amongst other components, a transmission circuitry and a reception circuitry. In FIG. 1B, the sensor may include antenna elements 302, which may (or may not) be shared between the transmission circuitry and the reception circuitry.

In various embodiments, a back portion of the occupant 110 is facing the antenna elements 302. The antenna elements 302 may be positioned within the vehicle seat 120, on the front (i.e., portion of the vehicle seat 120 near the occupant 110), or on the back (i.e., portion of the vehicle seat 120 away from the occupant 110). Further, the antenna elements 302 may be integrated uniformly within the vehicle seat 120 or selectively positioned in a top, bottom, center, left, or right position of the vehicle seat 120. The antenna elements 302 may be flexible and conform to the general outline of the vehicle seat 120. The antenna elements 302 may be arranged in an array configuration. As an example, the antenna elements 302 may be arranged in a star, matrix, circle, square, rectangle, diamond, or other patterns.

In order to improve measurement accuracy and reduce measurement errors, it is beneficial to reduce or minimize potential interfering and blocking elements between the antenna elements 302 and the rib cage 140 of the occupant 110. The placement of the antenna elements 302 to take measurements from the back of the occupant 110, advantageously minimizes obstructions caused by body parts (e.g., head and arm) while maintaining accuracy and reliability of the measurements.

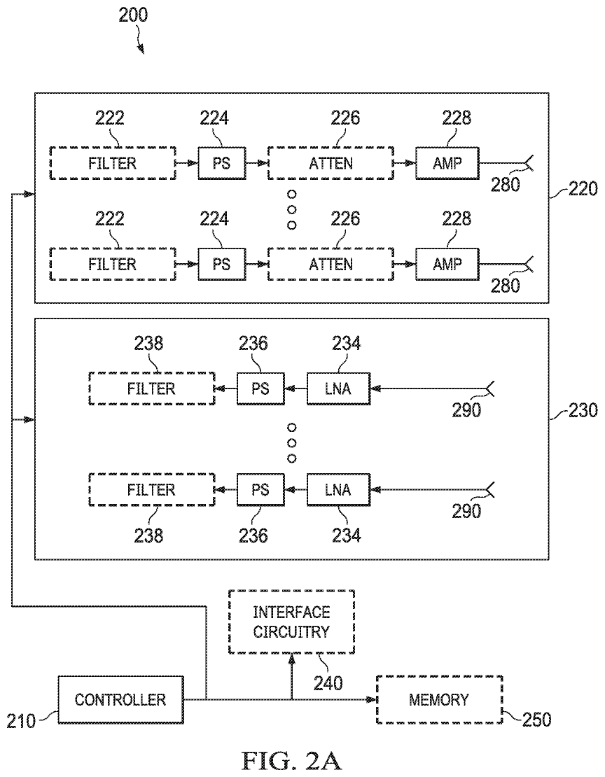

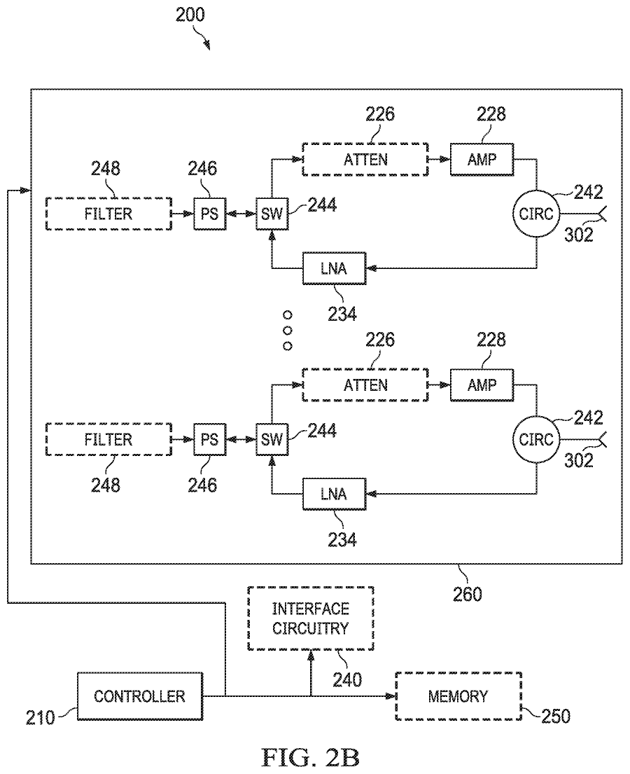

Typically, a vehicle seat 120 may have an area of about 40 square centimeters (cm²) with a depth of about 10 cm. Such a volume of space provides adequate room for the placement of multiple antenna elements 302 within the vehicle seat 120. As noted above, the antenna elements may be arranged across the height and width of the vehicle seat 120. FIG. 2A illustrates a system block diagram for an embodiment sensor 200 for measuring a vital sign of the occupant 110. The sensor 200 may use beamforming techniques to focus the radar beam to a specific point, for example the location of the heart within the rib cage of the occupant 110. As shown, the sensor 200 includes a controller 210, a transmission circuitry 220, and a reception circuitry 230, which may (or may not) be arranged as shown. Optionally, the sensor 200 may include an interface circuitry 240 and a memory 250. The sensor 200 may include additional components not depicted in FIG. 2, such as long term storage (e.g., non-volatile memory, etc.).

The controller 210 may be any component or collection of components adapted to perform computations and/or other processing related tasks. The controller 210 may be a microprocessor, a digital signal processor, a field programmable gate array (FPGA), an application specific integrated circuit (ASIC), or the like. In some embodiments, the controller 210 is external to the sensor 200 package. The controller 210 may execute the instructions to perform the steps of calibration, measurement, analysis, storage, and/or communication of the data associated with the vital sign measurements. In embodiments, the controller 210 may collect the raw data and communicate the data to be analyzed by an external processor. In other embodiments, the controller 210 may perform all or some steps related to the calibration, measurement, analysis, storage, and/or communication of the data associated with the vital sign measurements.

The transmission circuitry 220 is used to transmit a reconfigurable radar beam to the occupant 110. The transmission circuitry 220 may include one or more filters 222, phase shifters 224, attenuators 226, amplifiers 228, and antenna elements 280, which may (or may not) be arranged as shown. The amplifiers 228 may be high power amplifiers, multi-stage amplifiers, variable gain amplifiers, or the like.

The reception circuitry 230 is used to receive a reflected signal, which is a reflection of the RF transmission from the back side of the occupant 110. The reception circuitry 230 may include one or more antenna elements 290, amplifiers 234, phase shifters 236, and filters 238, which may (or may not) be arranged as shown.

Further, although in each of the transmission circuitry 220 and the reception circuitry 230, two RF paths are shown, less or more RF paths and components may be used based on the topology of the sensor 200.

The embodiments of this disclosure advantageously direct a radar beam to the back side of the occupant 110 and towards the location of the heart situated within the rib cage 140. This minimizes impediments between the rib cage 140 and the radar beam that may be caused by the occupant 110.

The controller 210, in communication with the transmission circuitry 220, may adjust the phase of an RF transmission to direct the beam towards the rib cage 140 using the phase shifters 224. The controller 210 may additionally adjust the amplitude of the RF transmission, using attenuators 226 and/or amplifiers 228 in the transmission circuitry 220, to provide a reliable and consistently accurate RF signal to account for changes in the size, shape, and movements of an occupant 110 of the vehicle 100. In some embodiments, the RF transmission is a continuous transmission. In other embodiments, the RF transmission is a pulsed transmission. In other embodiments, the RF transmission varies between a continuous and a pulse transmission. In some embodiments, the RF transmission is a modulated signal.

The interface circuitry 240 may be any component or collection of components that allow the sensor 200 to communicate with other devices/components and/or one or more users. In one embodiment, the interface circuitry 240 may be a display interface that is used to display the raw data, or analyzed data, based on the measured vital signs to the occupant 110 or a different user. In another embodiment, the interface circuitry 240 may include a telecommunications transceiver adapted to transmit and receive signaling over a wireless or a wireline telecommunication network within the vehicle 100 and/or to a networked component external to the vehicle 100. The external component may be a cloud based or cellular based application that is used to display the data to the occupant 110 or to share the data with another user or an emergency care specialist.

The memory 250 may be any component or collection of components adapted to store programming and/or instructions for execution by the controller 210. In an embodiment, the memory 250 includes a non-transitory computer readable medium. In embodiments, the memory 250 may optionally store calibration data. In an embodiment, the memory 250 may be used to store raw data collected by the sensor 200. In another embodiment, the memory 250 may be used to store processed data based on the raw data collected by the sensor 200 and analyzed by the controller 210 or another processing component, such as a cloud based processing system.

FIG. 2B illustrates a system block diagram for another embodiment sensor 200 for measuring a vital sign of the occupant 110. The embodiment sensor 200 in FIG. 2B shares some components as previously discussed with respect with FIG. 2A. However, the transceiver circuitry 260 in FIG. 2B include components that were separate in FIG. 2A.

The transmit path of the transceiver circuitry 260 may include a filter 248, phase shifter 246, RF switch 244, attenuator 226, amplifier 228, circulator 242, and antenna element 302, which may (or may not) be arranged as shown.

The receive path of the transceiver circuitry 260 may include the antenna element 302, circulator 242, LNA 234, RF switch 244, phase shifter 246, and filter 248.

The RF switch 244 may be controlled using the controller 210 to switch between the receive and transmit paths. The circulator 242 directs the RF signal based on the input port that the signal is received through the appropriate path and the antenna element 302.

The sensor 200 in FIG. 2B shares the antenna elements 302, the RF switches 244, the phase shifters 246, the filters 248, and the circulators 242 between the transmission paths and the reception paths of the circuit.

Similar to FIG. 2A, the transceiver circuitry 260 may include multiple transmit and reception paths, although two such paths are shown in FIG. 2B for simplicity of the discussion. FIGS. 2A-B illustrate schematic representations between the various circuitry components of non-limiting embodiment sensors. The components of the sensor 200 may be packaged within the vehicle seat 120 or may disparately located at various locations within the vehicle 100. As an example, a controller 210 of the sensor 200 may be located within the dashboard of the vehicle 100.

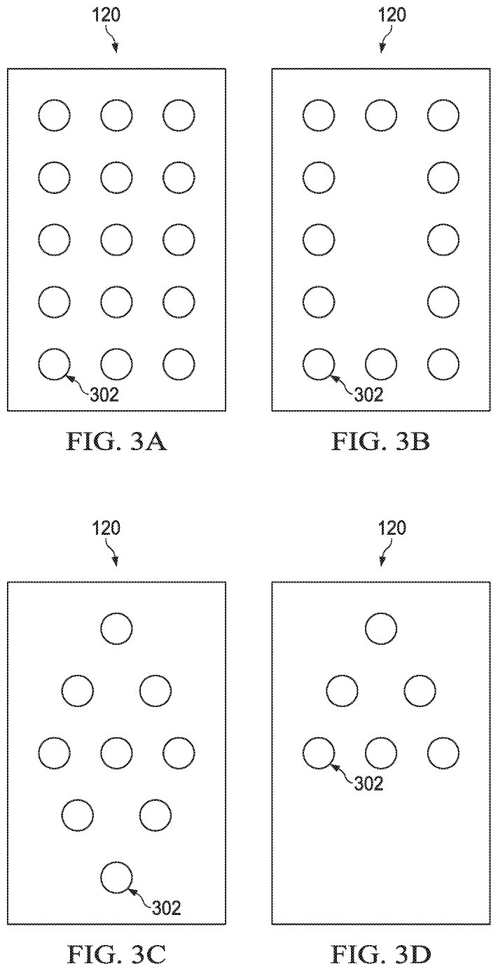

FIGS. 3A-D illustrate several non-limiting embodiment arrangements of antenna elements within a vehicle seat 120. In each of these Figures, the antenna elements 302 may be a transmitting antenna element 280, a receiving antenna element 290, or a common antenna element shared between the transmission circuitry 220 and the reception circuitry 230. The arrangements shown in these Figures are examples and do not limit the many different type of arrangement (e.g., diamond, circle, matrix, uneven spread, focused at a certain location, etc.) of the antenna elements 302. The antenna elements 302 may be flexible or non-flexible. The antenna elements 302 may be located in the headrest or the back seat portion of the vehicle seat 120. The antenna elements 302 may be located in-between the vehicle seat 120 and the occupant 110, within the vehicle seat 120, or on the back side of the vehicle seat 120 and away from the occupant 110 (i.e., the vehicle seat 120 is located between the antenna elements 302 and the occupant 110).

FIG. 3A illustrates a grid-like positioning of the antenna elements 302 around the vehicle seat 120. FIG. 3B illustrates a rectangular configuration of the antenna elements 302 within the vehicle seat 120. FIG. 3C illustrates an elliptical configuration of the antenna elements 302 with an antenna element 302 located at the center of the vehicle seat 120. FIG. 3D illustrates a non-homogenous placement of the antenna elements 302 focused on the upper portion of the vehicle seat 120.

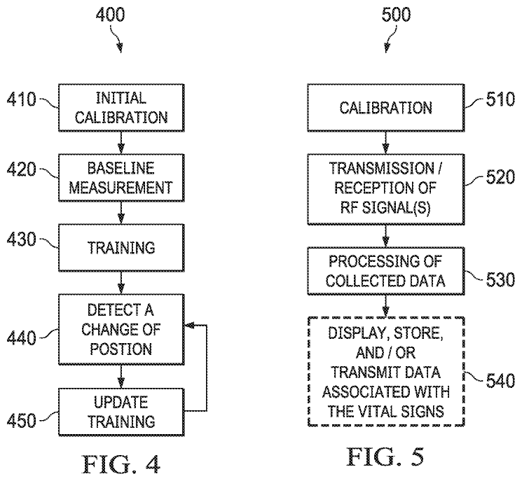

FIG. 4 is a flowchart of an embodiment method 400 for calibrating the sensor 200, which may be performed by controller 210. The calibration of the sensor 200 may improve the accuracy of the vital sign measurements in view of the potential ambient signals that can introduce errors into the data. In other words, a benefit of the calibration procedure is to improve the signal to noise ratio (SNR) of the measurements. Further, the calibration procedure provides a reliable and accurate method for extracting a signature, heartbeat, and/or respiratory rate of an occupant of a vehicle seat based on movements of the occupants rib cage.

In an embodiment, a received pulse can be affected by an antenna's transfer functions in both transmit and receive operating modes. As an example, mutual coupling between the antennas and/or the scattering from a target, or nearby objects, can influence the received pulse. As another example, a transmitting antenna may time-differentiate an input pulse during transmission. It is therefore advantageous to eliminate, or reduce, external effects from the received pulse to extract a target signature.

A received pulse can be mathematically modelled as: sᵣ(t)=sₜ(t)*h_{med}(t)*h_{Tx}(t)*h_{Rx}(t)+sₜ(t)*h_{med}(t)*h_{Tx}(t)*h_{Rx}(t)*h_{tgt}(t). In which sᵣ(t) and sₜ(t) are, respectively, the receive and transmit pulses; and h_{med}(t), h_{Tx}(t), h_{Rx}(t), and h_{tgt}(t) are, respectively, the target impulse response, the antenna impulse response in transmit mode, the antenna impulse response in receive mode, and the medium impulse response. The symbol (*) represents the convolution operator in the time domain. The first part of the equation above corresponds to the antenna's mutual coupling in the medium of propagation, which can be unbounded or bounded regions filled with any material(s). The second part of the equation above corresponds to the target signature. In order to obtain the target signature and calibrate the pulse s_{cat}(t), the ambient pulse, sₐₘ(t), has to be removed: s_{cat}(t)=sᵣ(t)−sₐₘ(t).

The ambient pulse is the effect of everything with the exception of the target, which includes measurement instruments and surrounding objects. An ambient pulse can be measured by transmitting and receive a pulse in the medium of propagation, in the absence of a target. In an embodiment, a calibrated pulse contains the reflected pulse from the target with the effects of ambient removed from the measurement.

At step 410, the controller 210 performs an initial calibration of the sensor 200. The initial calibration is used to determine an ambient pulse that is removed from the real-time measurements after system calibration. In other words, the initial calibration allows an extraction of a target signature (i.e., vital sign measurements). The ambient pulse is a received pulse that is measured in an absence of the occupant 110 and used to capture the effect of the surrounding medium (i.e., vehicle parts).

In an embodiment, a pulse is sent from the transmission circuitry 220 in the absence of the occupant 110 of the vehicle 100. This step may be performed at any time that the vehicle seat 120 is not occupied by the occupant 110. The data collected (i.e., Sam(t)) from this ambient pulse is subtracted from data collected (i.e., Sr(t)) in the presence of the occupant 110 to provide a calibrated measurement: Scal(t)=Sr(t)−Sam(t). This advantageously helps to remove unwanted effects, such as reflections from the seat, cables, dashboard, cables, or mutual coupling from the multitude of antennas in the sensor 200.

At step 420, the controller 210 performs a baseline measurement. The baseline measurement is performed after the initial calibration and to improve the validity of the vital sign measurements. In this step, the sensor 200 performs multiple rounds of measurements in the presence of the occupant 110. The repetition of the measurements provide an opportunity to reduce the error and stables the settings record stable and valid signal measurements. The baseline measurement includes the training of the sensor until the output of the training provides a converged data set with minimal errors. The convergence may be a defined limit, for example 2 errors in the heartbeat per minute.

The data for the initial calibration and baseline measurements may be stored in the memory 250 or in an external memory located within or external to the vehicle 100.

At step 430, the controller 210 is trained to steer one or more radar beams provided by the transmission circuitry 220 towards the location of the heart within the rib cage 140 of the occupant 110 of the vehicle 100. As various occupants of the vehicle may have varying body types, sizes, and shapes, the training of the controller 210 advantageously directs the radar beam to be pointed to the heart regardless of preconfigured settings. The steering of the beam(s) to the heart location improves the collection of the vital sign measurements and increases the signal to noise ratio of the collected data.

In an embodiment, to direct the reconfigurable radar beam towards the location of the heart, a silhouette of the occupant may be determined. The determination of the boundaries of the body may be performed by steering the reconfigurable radar beam at various directions by changing the phase shifters of the sensor 200. The controller 210 may then use the reflected data to determine the shape, size, and overall silhouette of the human occupying the vehicle seat. In some embodiments, the controller 210 may then direct the radar beam, based on the silhouette, towards a typical location of the heart (e.g., upper left portion of the body when the sensor is located behind the occupant) within the rib cage. In one embodiment, the controller 210 may additionally, or alternatively, hone into the location of the heart based on an increase in the signal to noise ratio of heart beats collected during the calibration procedure.

Advantageously, multiple antenna elements, configured to generate a beam steered antenna radiation pattern, provide an opportunity to vary the direction of the main lobe of the antenna radiation pattern to scan across the volume around the vehicle seat. The contrast in dielectric constant between the human body and the surrounding elements provides a suitable means to differentiate between various reflected signals. The reflected signal from the human body tissue, for example having a higher dielectric constant, will have a different amplitude in contrast with the reflected signal from the other non-human elements. In an embodiment, the difference in the reflection signal by scanning across the volume around the sensor may be used to generate, for example, a silhouette of the human body.

At step 440, the controller 210 monitors movements of the occupant. In the event that the controller 210 detects a change in the positioning of the occupant 110 relative to the sensor 200, at step 450, the controller 210 updates the training and redirects the reconfigurable radar beam to point the radar beam towards the new location of the heart relative to the sensor 200. In some embodiments, one or more antenna elements of the sensor 200 can be dedicated to detecting a change in the body movement and positioning of the occupant 110. In one embodiment, several sensors around the vehicle seat 120 may be used to detect a change in the movement of the occupant 110, for example through the use of pressure or vicinity sensors. The steps 440 and 450 are repeated in the event that the controller 210 detects any further changes in the positioning of the occupant 110 relative to the antenna elements of the sensor 200.

It should be noted that although the method described hereinabove is performed by the controller 210, other means of processing, as further detailed in this disclosure, may perform these calibration steps. As an example, the calibration steps may be performed by a cloud based processor, the vehicle computer, a cellular phone, or the like.

FIG. 5 is a flowchart of an embodiment method 500 for collecting data associated with a vital sign measurement of an occupant no, as may be performed by a controller 210 of a sensor 200 located in a vehicle seat 120. At step 510, the controller 210 is calibrated in accordance with, for example, the method as described with respect to FIG. 4. The calibration procedure provides a real-time correction based on the movement of the occupant 110 relative to the antenna elements of the sensor 200 during the measurement collection. In addition, the calibration procedure improves the targeting of the heart location by providing an improved method for beam steering of the RF transmission and reception of the reflected RF signal to account for any changes in the positioning of the occupant 110.

At step 520, the controller 210 uses the calibration data to transmit an RF transmission and receive a reflected RF signal from the occupant 110 of the vehicle 100. This is performed by adjusting the various phase shifters, attenuators, and amplifiers within the transmission circuitry 220 and reception circuitry 230. For each antenna element, the phase and/or time delay adjustment may be based on a desired direction of the main lobe (i.e., main beam) of the antenna radiation pattern formed by the transmission circuitry. In an embodiment, a summation (SUM)/Difference radiation pattern is used to track a target. In such an embodiment, the alignment of the peak and the null of the radiation pattern is used to provide a target direction. A comparison of the location of the target with the reference point (e.g., heart location) provides an updated phase and/or time-delay that is used to adjust the direction of the antenna radiation pattern. Similar methods and system algorithms may be used to advantageously beam steer the antenna radiation pattern to a desired target.

One or more RF signals are transmitted towards the location of the heart within the ribcage of the occupant 110. The reflection of the RF signals is received by the reception circuitry 230.

In an embodiment, the controller 210 may optionally notify the occupant 110 to adjust his/her posture to improve the collection of the vital sign measurements. A corresponding feedback signal may be signaled to the occupant 110 using, for example, a display mounted on the dashboard or on the steering wheel 150, a cellular phone, or a signaling device. In an embodiment, the controller 210 may determine a drop in the signal to noise ratio of the vital sign measurements. Accordingly, the feedback signal may instruct or request the occupant 110 to reposition his/her body relative to the vehicle seat 120. The feedback signal may continue to request a repositioning of the occupant 110 until the data, corresponding to the vital signs, meet a threshold requirement.

Optionally at step 530, the controller 210 processes the data associated with the vital sign measurements. In some embodiments, however, the processing of the vital sign data may be performed by an external component to the sensor 200, such as the vehicle computer, a cellular phone or tablet, or a cloud based processor. In some embodiments, the data associated with the vital sign is used in combination with data collected from one or more external sources, such as a wearable device (e.g., smart watch, chest strap, wrist strap, etc.) to provide a more comprehensive collection of vital sign measurements to the occupant or another user.

In an embodiment, the reflected pulses are measured and calibrated. The length of the measurement is such that the rib cage movements, as a result of breathing and heartbeat, are sufficiently captured. The collected set of measurements includes various amplitude levels and time based information (e.g., time delay) tied to, for example, the radar cross-section change due to of the movements of the ribcage over time. A low pass filter may be used to isolate the vital sign measurements that operate at relative low frequencies and to remove unwanted high frequency components. Various methods, such as fast Fourier transformation, may be used for these purposes.

Optionally at step 540, the data associated with the vital sign is displayed to the occupant 110 or to another user, or alternatively stored in a memory of the vehicle computer, the sensor 200, a mobile computer, a cloud based storage, or the like. As an example, the data may be displayed on a dashboard of the vehicle 100. As another example, the data may be provided wirelessly to an emergency care unit.

In some embodiments, vital signal processing methods may be used that are disclosed in U.S. patent application Ser. No. 15/872,677, which has been incorporated by reference herein in its entirety.

Although the description has been described in detail, it should be understood that various changes, substitutions, and alterations may be made without departing from the spirit and scope of this disclosure as defined by the appended claims. The same elements are designated with the same reference numbers in the various figures.

Moreover, the scope of the disclosure is not intended to be limited to the particular embodiments described herein, as one of ordinary skill in the art will readily appreciate from this disclosure that processes, machines, manufacture, compositions of matter, means, methods, or steps, presently existing or later to be developed, may perform substantially the same function or achieve substantially the same result as the corresponding embodiments described herein. Accordingly, the appended claims are intended to include within their scope such processes, machines, manufacture, compositions of matter, means, methods, or steps.

The specification and drawings are, accordingly, to be regarded simply as an illustration of the disclosure as defined by the appended claims, and are contemplated to cover any and all modifications, variations, combinations, or equivalents that fall within the scope of the present disclosure.

While this invention has been described with reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

Example 1 is a sensor that includes a transmission circuitry, a reception circuitry, and a controller. The transmission circuitry is configured to transmit a radar beam to an occupant in a vehicle seat. The reception circuitry is configured to receive a reflected signal-a reflection of the radar beam from the occupant. The controller is configured to determine data associated with a vital sign of the occupant based on the reflected signal.

In example 2, the subject matter of example 1 can optionally further include an interface circuitry configured to transmit data from and receive data by the sensor.

In example 3, the subject matter of example 1 or 2 can optionally further include a controller configured to transmit the data associated with the vital sign of the occupant to an external component using the interface circuitry.

In example 4, the subject matter of examples 1 to 3, where the vital sign is a heart rate, a respiratory rate, or a blood pressure rate of the occupant, or a combination thereof.

In example 5, the subject matter of examples 1 to 4 can optionally further include a non-transitory memory storage configured to store the data associated with the vital sign of the occupant.

In example 6, the subject matter of examples 1 to 5, where the reception circuitry comprises a plurality of antennas arranged in an array configuration within a seat portion of a vehicle seat assembly.

In example 7, the subject matter of examples 1 to 6, where the transmission circuitry comprises a plurality of antennas arranged in an array configured within a seat portion of a vehicle seat assembly.

In example 8, the subject matter of examples 1 to 7, where the controller is further configured to calibrate the transmission circuitry to direct the radar beam to a heart location on a rib cage of the occupant.

In example 9, the subject matter of examples 1 to 8, where the transmission circuitry is configured to direct the radar beam to a posterior upper limb portion of the occupant.

In example 10, the subject matter of examples 1 to 9, where the controller is further configured to determine a position of the occupant in the vehicle seat based on an estimated time delay, an amplitude, or a phase of the reflected signal and, based thereon, to adjust one or more phase shifters in the transmission circuitry.

In example 11, the subject matter of examples 1 to 10, where the controller is further configured to determine a position of the occupant in the vehicle seat based on an estimated time delay, an amplitude, or a phase of the reflected signal and, based thereon, to adjust one or more phase shifters in the reception circuitry.

In example 12, the subject matter of examples 1 to 11, where the radar beam includes a continuous RF signal, a pulsed RF signal, a modulated RF signal, or a combination thereof.

Example 13 is a method that includes transmitting, by a transmission circuitry, a radar beam to an occupant in a vehicle seat; receiving, by a reception circuitry, a reflected signal that is a reflection of the radar beam from the occupant; and determining, by a controller, data associated with a vital sign of the occupant based on the reflected signal.

In example 14, the method of example 13 optionally further includes transmitting, using an interface circuitry, the data associated with the vital sign of the occupant to an external component.

In example 15, the method of examples 13 or 14 optionally further includes analyzing, storing, or displaying the data associated with the vital sign of the occupant, or a combination thereof.

In example 16, the method of examples 13 to 15 optionally further includes calibrating the transmission circuitry to direct a direction of the radar beam to a rib cage of the occupant.

In example 17, the method of examples 13 to 16 optionally further includes determining a position of the occupant in the vehicle seat based on an estimated time delay, an amplitude, or a phase of the reflected signal and, based thereon, adjusting one or more phase shifters in the transmission circuitry.

In example 18, the method of examples 13 to 17 optionally further includes determining a position of the occupant in the vehicle seat based on an estimated time delay, an amplitude, or a phase of the reflected signal and, based thereon, adjusting one or more phase shifters in the reception circuitry.

In example 19, the method of examples 13 to 18 optionally further includes determining a position of the occupant in the vehicle seat based on an estimated time delay, an amplitude, or a phase of the reflected signal and, based thereon, providing a feedback to the occupant to adjust his/her posture for improved measurement accuracy.

In example 20, the method of examples 13 to 19, where the vital sign is a first vital sign, and the method of examples 13 to 19 optionally further includes receiving, from an external source, a second vital sign of the occupant and, based thereon, determining a combined data associated with the second vital sign and the first vital sign.

In example 21, the method of examples 13 to 20, where the radar beam comprises a continuous RF signal, a pulsed RF signal, a modulated RF signal, or a combination thereof.

Example 22 is a vehicle seat that includes a seat portion; a non-transitory memory storage; a plurality of antennas configured in an array configuration within the seat portion; a transmission circuitry coupled with the plurality of antennas, the transmission circuitry configured to transmit, over one or more of the plurality of antennas, a radar beam to an occupant in the vehicle seat; a reception circuitry coupled with the plurality of antennas, the reception circuitry configured to receive, over one or more of the plurality of antennas, a reflected signal that is a reflection of the radar beam from the occupant; and a controller configured to: determine data associated with a vital sign of the occupant based on the reflected signal; and store the data associated with the vital sign of the occupant in the non-transitory memory storage.

In example 23, the subject matter of example 22, where the controller is further configured to determine occupancy of the vehicle seat by the occupant and, based thereon, transmitting the radar beam.

In example 24, the subject matter of examples 22 or 23, where a plurality of N number of antennas are arranged in a generic pattern configuration, wherein an M number of antennas are transmitting antennas and an (N-M) number of antennas are receiving antennas, N being a positive integer greater than 1 and M being a positive integer.

In example 25, the subject matter of examples 22 to 24, where the plurality of antennas are arranged in a generic pattern configuration, wherein each antenna is a transmitting antenna and a receiving antenna.

In example 26, the subject matter of examples 22 to 25 optionally further includes a plurality of circulators, wherein the transmission circuitry and the reception circuitry are coupled to each antenna using a respective circulator.

In example 27, the subject matter of examples 22 to 26, where the controller is further configured to determine a change in a posture of the occupant in the vehicle seat and, based thereon, providing feedback, using an external display on a vehicle that the vehicle seat is mounted, for the occupant to adjust the posture of the occupant for improved vital sign measurements.

In example 28, the subject matter of examples 22 to 27, where the controller is further configured to provide, to an external component on a vehicle that the vehicle seat is mounted, data associated with the vital sign to be displayed by the external component.

In example 29, the subject matter of examples 22 to 28, where the radar beam comprises a continuous RF signal, a pulsed RF signal, a modulated RF signal, or a combination thereof.

What is claimed is:

1. A sensor, comprising:
a transmission circuitry configured to transmit a radar beam to an occupant in a vehicle seat;
a reception circuitry configured to receive a reflected signal, the reflected signal being a reflection of the radar beam from the occupant; and
a controller configured to determine data associated with a vital sign of the occupant based on the reflected signal.

2. The sensor of claim 1, further comprising an interface circuitry configured to transmit data from and receive data by the sensor.

3. The sensor of claim 2, wherein the controller is further configured to transmit the data associated with the vital sign of the occupant to an external component using the interface circuitry.

4. The sensor of claim 1, wherein the vital sign is a heart rate, a respiratory rate, or a blood pressure rate of the occupant, or a combination thereof.

5. The sensor of claim 1, further comprising a non-transitory memory storage configured to store the data associated with the vital sign of the occupant.

6. The sensor of claim 1, wherein the reception circuitry comprises a plurality of antennas arranged in an array configuration within a seat portion of a vehicle seat assembly.

7. The sensor of claim 1, wherein the transmission circuitry comprises a plurality of antennas arranged in an array configured within a seat portion of a vehicle seat assembly.

8. The sensor of claim 1, wherein the controller is further configured to calibrate the transmission circuitry to direct the radar beam to a heart location on a rib cage of the occupant.

9. The sensor of claim 1, wherein the transmission circuitry is configured to direct the radar beam to a posterior upper limb portion of the occupant.

10. The sensor of claim 1, wherein the controller is further configured to determine a position of the occupant in the vehicle seat based on an estimated time delay, an amplitude, or a phase of the reflected signal and, based thereon, to adjust one or more phase shifters in the transmission circuitry.

11. The sensor of claim 1, wherein the controller is further configured to determine a position of the occupant in the vehicle seat based on an estimated time delay, an amplitude, or a phase of the reflected signal and, based thereon, to adjust one or more phase shifters in the reception circuitry.

12. The sensor of claim 1, wherein the radar beam comprises a continuous RF signal, a pulsed RF signal, a modulated RF signal, or a combination thereof.

13. A method, comprising:
transmitting, by a transmission circuitry, a radar beam to an occupant in a vehicle seat;
receiving, by a reception circuitry, a reflected signal that is a reflection of the radar beam from the occupant; and
determining, by a controller, data associated with a vital sign of the occupant based on the reflected signal.

14. The method of claim 13, further comprising transmitting, using an interface circuitry, the data associated with the vital sign of the occupant to an external component.

15. The method of claim 13, further comprising analyzing, storing, or displaying the data associated with the vital sign of the occupant, or a combination thereof.

16. The method of claim 13, further comprising calibrating the transmission circuitry to direct a direction of the radar beam to a rib cage of the occupant.

17. The method of claim 13, further comprising determining a position of the occupant in the vehicle seat based on an estimated time delay, an amplitude, or a phase of the reflected signal and, based thereon, adjusting one or more phase shifters in the transmission circuitry.

18. The method of claim 13, further comprising determining a position of the occupant in the vehicle seat based on an estimated time delay, an amplitude, or a phase of the reflected signal and, based thereon, adjusting one or more phase shifters in the reception circuitry.

19. The method of claim 13, further comprising determining a position of the occupant in the vehicle seat based on an estimated time delay, an amplitude, or a phase of the reflected signal and, based thereon, providing a feedback to the occupant to adjust his/her posture for improved measurement accuracy.

20. The method of claim 13, wherein the vital sign is a first vital sign, the method further comprising receiving, from an external source, a second vital sign of the occupant and, based thereon, determining a combined data associated with the second vital sign and the first vital sign.

21. The method of claim 13, wherein the radar beam comprises a continuous RF signal, a pulsed RF signal, a modulated RF signal, or a combination thereof.

22. A vehicle seat, comprising:
a seat portion;
a non-transitory memory storage;
a plurality of antennas configured in an array configuration within the seat portion;
a transmission circuitry coupled with the plurality of antennas, the transmission circuitry configured to transmit, over one or more of the plurality of antennas, a radar beam to an occupant in the vehicle seat;
a reception circuitry coupled with the plurality of antennas, the reception circuitry configured to receive, over one or more of the plurality of antennas, a reflected signal that is a reflection of the radar beam from the occupant; and
a controller configured to:
determine data associated with a vital sign of the occupant based on the reflected signal; and
store the data associated with the vital sign of the occupant in the non-transitory memory storage.

23. The vehicle seat of claim 22, wherein the controller is further configured to determine occupancy of the vehicle seat by the occupant and, based thereon, transmitting the radar beam.

24. The vehicle seat of claim 22, wherein a plurality of N number of antennas are arranged in a generic pattern configuration, wherein an M number of antennas are transmitting antennas and an (N−M) number of antennas are receiving antennas, N being a positive integer greater than 1 and M being a positive integer.

25. The vehicle seat of claim 22, wherein the plurality of antennas are arranged in a generic pattern configuration, wherein each antenna is a transmitting antenna and a receiving antenna.

26. The vehicle seat of claim 22, further comprising a plurality of circulators, wherein the transmission circuitry and the reception circuitry are coupled to each antenna using a respective circulator.

27. The vehicle seat of claim 22, wherein the controller is further configured to determine a change in a posture of the occupant in the vehicle seat and, based thereon, providing feedback, using an external display on a vehicle that the vehicle seat is mounted, for the occupant to adjust the posture of the occupant for improved vital sign measurements.

28. The vehicle seat of claim 22, wherein the controller is further configured to provide, to an external component on a vehicle that the vehicle seat is mounted, data associated with the vital sign to be displayed by the external component.

29. The vehicle seat of claim 22, wherein the radar beam comprises a continuous RF signal, a pulsed RF signal, a modulated RF signal, or a combination thereof.
